(19) United States
(12) Patent Application Publication
(10) Pub. No.: US 2007/0208419 A1
(43) Pub. Date: Sep. 6, 2007

(54) Bifurcation stent with uniform side branch projection

(75) Inventors: Michael P. Meyer, Richfield, MN (US); Daniel Gregorich, St. Louis Park, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 11/368,964

(22) Filed: Mar. 6, 2006

(51) Int. Cl. A61F 2/06

(52) U.S. Cl. 623/1.35

(57) ABSTRACT

An expandable stent may be made having a tubular body and an expandable side branch section. The tubular body has at least one perimeter which defines at least one opening in the wall of the tubular body. The expandable side branch section has a plurality of elongate members with a first end engaged to the perimeter. Each of the plurality of elongate members has a second end that extends to a pre-determined distance away from the tubular body when in an expanded state. The expandable side branch section is configured to expand outward to allow for the formation of a bifurcated stent with a side branch.

### CROSS-REFERENCE TO RELATED APPLICATIONS

Not Applicable

### STATEMENT REGARDING FEDERALLY SPONSORED RESEARCH

### Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The present invention includes many different embodiments. Various embodiments of the invention are directed to designs of stents, bifurcated stents and/or the methods utilized to deliver a bifurcated stent to a bifurcation site.

In one or more embodiments, the invention is directed to an expandable stent having a flow path therethrough and an exterior surface and an interior surface, a constant inner diameter and an outer diameter. The stent comprises an expandable tubular body and an expandable side branch section having a plurality of elongate members. The expandable tubular body has a perimeter member which defines an opening. The opening has a center point located at a first longitudinal coordinate and a first circumferential coordinate. Each elongate member of the side branch section has a first end and a second end. The first end is engaged to the perimeter member of the tubular body. In an unexpanded state, the second end is positioned closer to the center point than the first end. In an expanded state, the second end of each elongate member extends to a pre-determined distance away from the tubular body of the stent. Each elongate member of the side branch section may have a triangular, columnar or zig-zag shape. The stent has no more than one side branch section disposed about a center point located at the first longitudinal coordinate.

The invention is also directed to bifurcated stents formed by providing any of the expandable stents disclosed herein, expanding the tubular body of the stent to a first diameter and expanding the plurality of elongate members outward to define a second flowpath which branches off the longitudinal flowpath and is in fluid communication therewith.

The invention is also directed to, in combination, a catheter and an expandable stent having a tubular surface disposed about a longitudinal flowpath. The tubular surface includes a plurality of elongate members which are arranged along the surface about an opening having a first size in an unexpanded state of the stent. The plurality of elongate members arranged so that the second ends of the elongate members extend to a uniform distance into the side branch vessel when the side branch section is expanded. The expandable stent is disposed about the catheter which includes an elongated catheter member which extends through the expandable side branch section.

These and other embodiments which characterize the invention are pointed out with particularity in the claims annexed hereto and forming a part hereof. However, for further understanding of the invention, its advantages and objectives obtained by its use, reference can be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIG. 1a is a side view of the stent with a tubular body and a side branch section in an unexpanded state, the side branch section in this embodiment has four elongate members.

FIG. 1b is a side view of an alternative embodiment of FIG. 1a where each of the four elongate members of the side branch section has an opening within the body of the elongate member.

FIG. 2a is a side view of the stent in FIG. 1b with both the tubular body and the side branch section in an expanded state.

FIG. 2b is an end view of the stent in FIG. 1b with both the tubular body and the side branch section in an expanded state.

FIG. 3a is a side view of the side branch section in an unexpanded state, the side branch section in this embodiment has twelve elongate members.

FIG. 3b is a side view of an alternative embodiment of FIG. 3a where each of the twelve elongate members of the side branch section has an opening within the body of the elongate member.

FIG. 4 is a side view of the side branch section in an unexpanded state, the side branch section in this embodiment has four elongate members with a zig-zag shape.

FIG. 5a is a side view of the tubular body where the elongate members of the side branch opening have a zig-zag shape and the interconnected serpentine bands forming the tubular body of the stent form the perimeter member.

FIG. 5b is a side view of the tubular body where the elongate members of the side branch opening have curved and straight sections and the interconnected serpentine bands forming the tubular body of the stent form the perimeter member.

FIG. 6 is an illustration of a method to determine the length of an elongate member with a zig-zag shape.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

As used herein the term ‘stent’ refers to an expandable prosthesis for implantation into a body lumen or vessel and includes devices such as stents, grafts, stent-grafts, vena cava filters, expandable frameworks, etc.

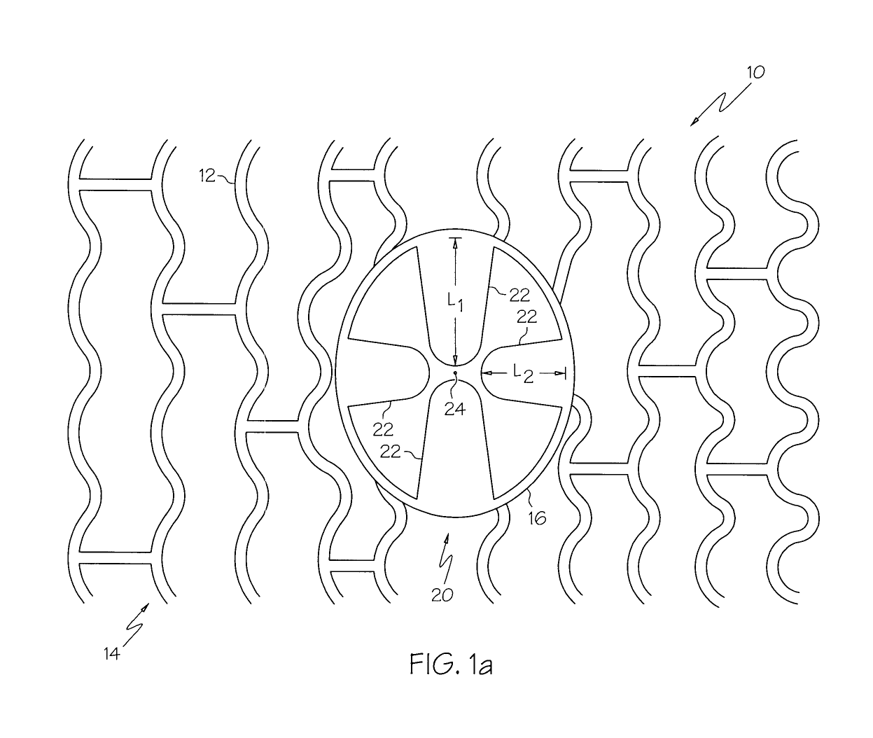

Referring now to the drawings which are for the purposes of illustrating embodiments of the invention only and not for purposes of limiting same, in at least one embodiment of the invention, an example of which is shown in side view in FIG. 1a, stent 10 comprises a tubular body 14 having a perimeter member 16 which defines an opening for an expandable side branch section 20. The opening has a center point 24 which is located at a first longitudinal coordinate and a first circumferential coordinate. The longitudinal coordinate indicates where, along the longitudinal length of the stent 10, the center point 24 is located. The circumferential coordinate indicates where, about the circumference of the stent 10, the center point 24 lies. The opening also has a longitudinal diameter and a circumferential diameter. In this embodiment, the circumferential diameter is greater than the longitudinal diameter.

The side branch section 20 comprises a plurality of elongate members 22 disposed about the center point 24. In this embodiment, the side branch section 20 has four elongate members 22. It is also within the scope of the invention to have five, six, seven, eight, nine, ten, eleven, twelve or more elongate members. Each elongate member 22 has a first end and a second end. The first end is engaged to the perimeter member 16 of the tubular body 14. The second end is positioned closer to the center point 24 than the first end.

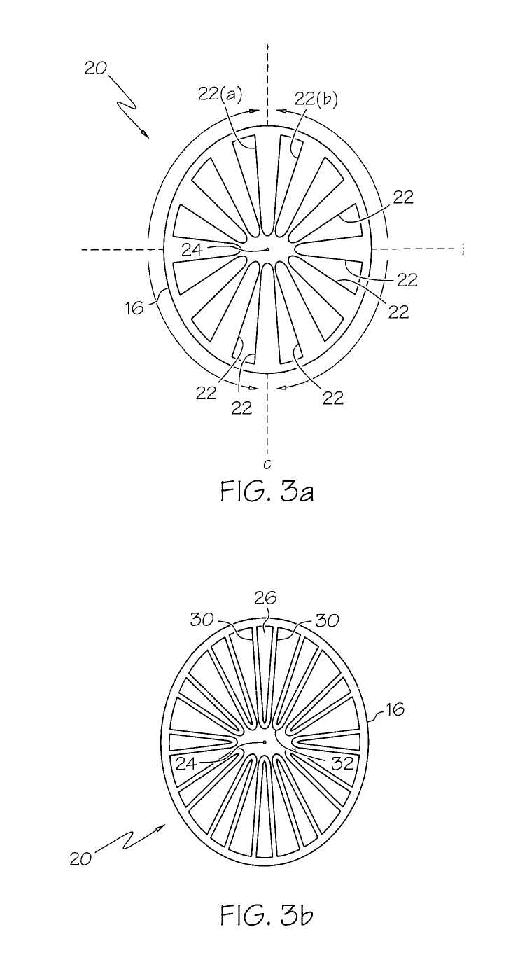

In at least one embodiment, shown in FIGS. 1b and 3b, the plurality of elongate members 22, that make up the side branch section 20, are not solid. In this embodiment, each elongate member 22 has two substantially straight sections 30 which are connected to each other by a curved section 32. Each of the two substantially straight sections 30 has a first end which is engaged to the perimeter member 16. The second end of the elongate member 22 is the curved section 32. The substantially straight sections 30 and the curved section 32 of the elongate member 22 define an opening 26.

Any suitable stent geometry may be used for the tubular body 14 of the stent 10. The pattern of interconnected serpentine bands 12 shown is shown by way of example only. The struts that form the serpentine band may be straight as shown in FIG. 1a or may be bent. In many of the figures, portions of the inventive stent are drawn without showing the structure of the tubular body of the stent or only the portion of the tubular body that defines the opening for the side branch section. It is understood that any suitable structure may be employed including, but not limited to, the cellular patterns, shown by way of example only, in U.S. Pat. No. 6,835,203, U.S. Pat. No. 6,348,065, and U.S. Pat. No. 6,013,091.

A non-bifurcated stent is formed when only the tubular body 14 is in an expanded state. A bifurcated stent is formed when both the tubular body 14 and the side branch section 20 are in an expanded state.

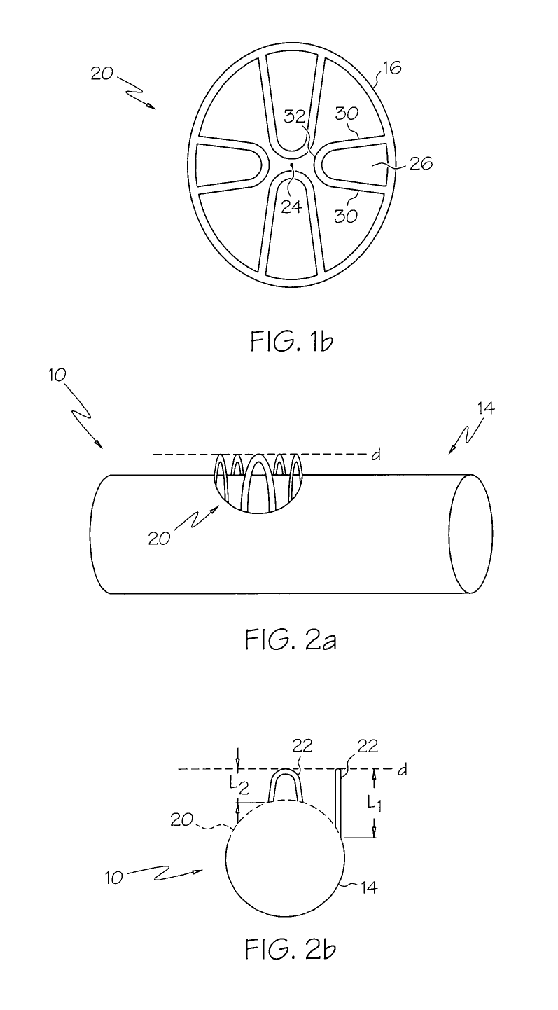

FIGS. 2a and 2b show two different views of the tubular body 14 and side branch section 20 of the stent 10 of FIG. 1b in an expanded state. When the elongate members 22 are expanded, the second ends of the elongate members extend to a pre-determined uniform distance (d) away from the tubular body 14 of the stent 10. In FIG. 2b, the apex of the curved section 32 is the second end of the elongate member 22 that extends to a pre-determined distance (d) away from the tubular body of the stent. When the stent is deployed in a body lumen like a vessel with a bifurcation, desirably this will provide greater coverage of the secondary lumen and a better region for the overlap of additional stents positioned in the secondary lumen. The side branch section 20 in an expanded state has a longitudinal axis which forms an oblique angle with the longitudinal axis of the tubular body 14. An oblique angle is any angle between 0-180 degrees and includes a 90 degree angle. In the embodiment of FIGS. 2a and 2b, the longitudinal axis of the side branch section 20 forms an angle with the longitudinal axis of the tubular body 14 that is substantially 90 degrees.

The invention is also directed to an expandable stent with an expandable side branch section having a plurality of elongate members disposed about a center point located at a first longitudinal coordinate and circumferential coordinate along the expandable stent. The longitudinal axis and the circumferential axis bisect the center point. Elongate members positioned on the circumferential axis are longer than elongate members positioned on the longitudinal axis. The length of the elongate members decreases as their position along the perimeter moves from the circumferential axis to the longitudinal axis and the length of the elongate members increases as their position along the perimeter moves from the longitudinal axis towards the circumferential axis.

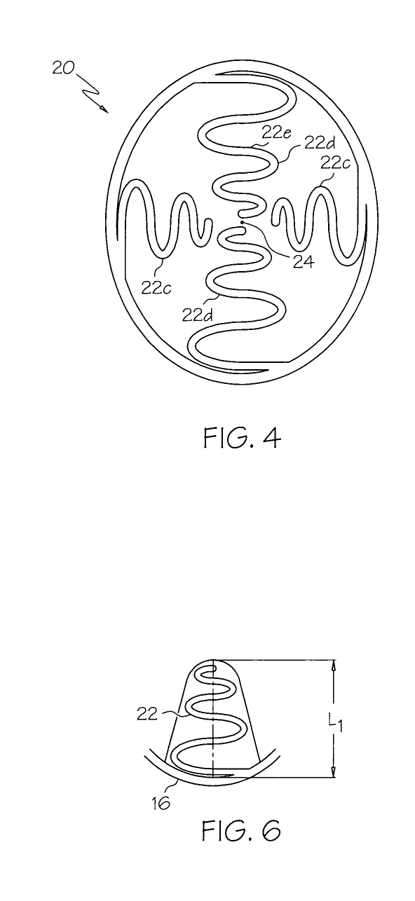

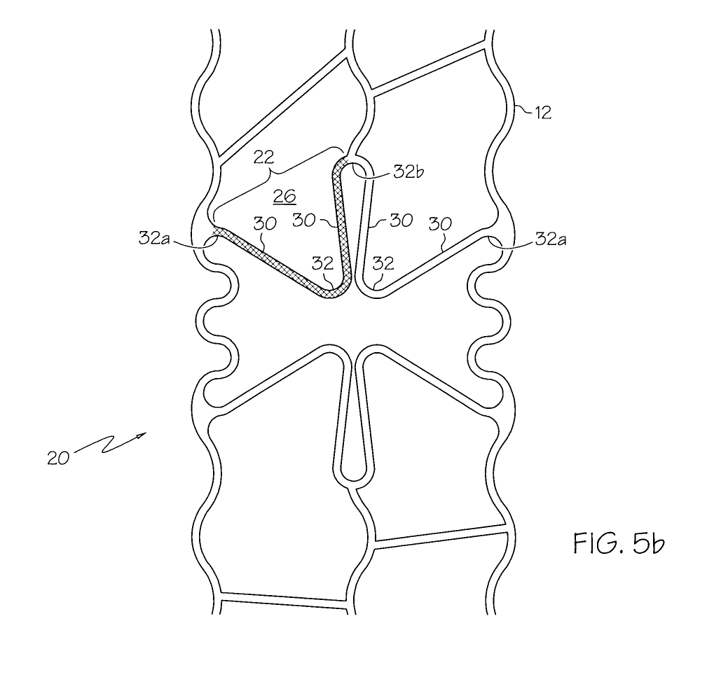

This can be seen in FIG. 1a where the elongate member 22 on the circumferential axis has a length L1 which is larger than the length L2 of the elongate member 22 on the longitudinal axis. As shown in FIG. 1a, the length of an elongate member is the distance measured in a straight line from the second end of the elongate member to perimeter member. Due to the curved nature of the elongate members 22 in FIGS. 1a and 1b, the apex of the curved second end is where the length of the elongate member is measured from. FIG. 6 shows an example of how the length of a zig-zag elongate member 22 can be determined. The zig-zag elongate member 22d of FIG. 4 is depicted in FIG. 6. A shape, similar to the elongate member 22 of FIG. 1a, has been drawn to encompass the zig-zag elongate member 22 and the length of the zig-zag elongate member 22 can then be determined as shown in FIG. 1a.

The arrows of FIG. 3a illustrate the change in length from the longitudinal axis (l) to the circumferential axis (c). The elongate member 22 with the shortest length is engaged to the perimeter member 16 where it intersects with the longitudinal axis (l) and is shown as being positioned at the beginning of the arrow. The elongate member 22 with the longest length is engaged to the perimeter member 16 where it intersects with the circumferential axis (c) and is shown as being positioned at the head of the arrow. Elongate members 22 engaged to the perimeter member 16 at positions between the longitudinal axis (l) and the circumferential axis (c) increase in length the closer their position on the perimeter member 16 is to the circumferential axis (c). This variation in length allows an elongate member positioned on the longitudinal axis to extend to the same distance away from the tubular body of the stent as an elongate member positioned on the circumferential axis even though the length of the elongate members themselves are different.

In many of the embodiments shown in the figures, FIG. 2 for example, there is no more than one side branch section located in a given circumferential section, or longitudinal coordinate, of the stent. Thus, there are no other side branch sections which are disposed about the circumference of the stent and located at the first longitudinal coordinate.

In other embodiments of the invention, additional side branch sections may be located within a given circumferential segment of the stent. The inventive stents may also have multiple side branch sections disposed along the length of the stent. Thus, additional side branch sections may be located at the first longitudinal coordinate or any other longitudinal and circumferential coordinate. In at least one embodiment, the stent has two side branch sections. In one embodiment, the two side branch sections are positioned at different longitudinal coordinates and the same circumferential coordinate. In one embodiment, the two side branch sections are positioned at different longitudinal coordinates and different circumferential coordinates. In one embodiment, the two side branch sections are positioned at the same longitudinal coordinate and different circumferential coordinates.

In another embodiment of the invention, shown in FIG. 3a, the side branch section 20 has twelve elongate members 22. Each of the twelve elongate members 22 of the side branch section 20 are disposed about a center point 24 of the side branch section 20. Some of the elongate members 22 have a more triangular shape 22(a), where the elongate member 22(a) tapers from the first end to the second end, while some of the elongate members 22 have a more columnar or rectangular shape 22(b), where the first and second ends of the elongate member 22(b) have substantially the same width.

It is within the scope of the invention for the elongate members to have many shapes, for example but not limited to, columnar or rectangular shapes, triangular shapes, or zig-zag shapes. In the embodiment shown in FIG. 3a, the elongate members 22 in the side branch section 20 have a plurality of shapes. In at least one embodiment, all the elongate members of a side branch section have the same shape. In the embodiment shown in FIG. 3a, the elongate members 22 are solid but in the embodiment shown in FIG. 3b, the elongate members 22 are not solid.

FIG. 4 shows an embodiment wherein the elongate members 22 of the side branch section 20 are zig-zag bands. The zig-zag elongate members 22 are disposed about a center point 24 of the side branch section 20. In this embodiment, the zig-zag elongate members have either four turns (22c) or five turns (22d) and each turn is connected to at least one substantially straight elongate member 22e. It is within the scope of the invention for the zig-zag elongate member 22 to have two, three, six, seven, eight, nine, ten, eleven, twelve or more turns.

The perimeter member of the tubular body defines the opening for the side branch section which can have any shape including, but not limited to, an oval, circular, or a rectangular shape, and can have any orientation so long as the elongate members forming the side branch extend to the same distance away from the tubular body of the stent when in an expanded state. In one embodiment, one of the elongate members has a length greater than the diameter of the opening so that in an unexpanded state, the second end (i.e. the end not engaged to the perimeter) extends across and slightly beyond the opening.

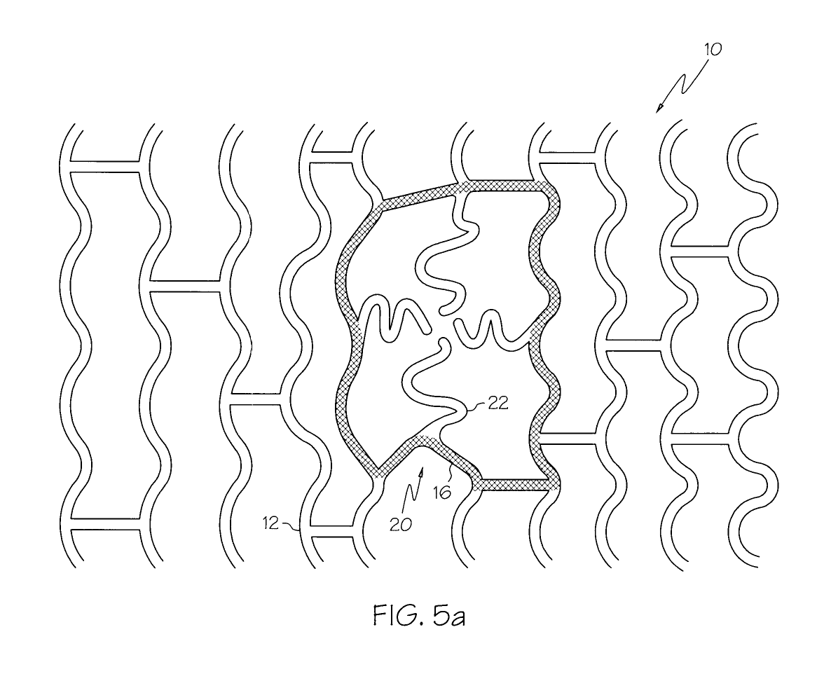

In FIG. 5a, the perimeter member 16 of the tubular body 14 is indicated by cross-hatching and defines an opening for the side branch section which has a rectangular shape. In addition, unlike FIGS. 1-4, the perimeter member 16 is composed of some of the interconnected serpentine bands 12 that form the tubular body instead of being a separate and distinct member from the interconnected serpentine bands 12. Although the opening defined by the perimeter member 16 has a different shape than pictured in FIG. 4, the zig-zag elongate members 22 have the characteristics of the inventive side branch 20, namely the circumferential elongate members have a greater length than the longitudinal elongate members thereby allowing all of the elongate members to extend to a predetermined distance away from the tubular body of the stent in the expanded state. In this embodiment, all the zig-zag elongate members 22 have three turns.

FIG. 5b shows another embodiment where the perimeter member is composed of some of the interconnected serpentine bands 12 of the tubular body. As shown in FIG. 5, elongate members 22 of the side branch section 20 are engaged to the interconnected serpentine bands 12 by the partial curved elongate members 32a and 32b. In this embodiment, there are four elongate members 22, one elongate member 22 is shown by hatchmarks. Each elongate member 22 has two straight members 30, a curved member 32, one partial curved member 32a engaged to the interconnected serpentine bands 12 and one partial curved member 32b that is engaged to an adjoining elongate member 22. Elongate member 22 and the interconnected serpentine bands 12 define an opening 26.

Any of the inventive stents disclosed herein may have a uniform inner diameter and/or a uniform outer diameter in the unexpanded state and/or in an expanded state. The inventive stents disclosed herein may also be provided in an embodiment in which the inner and/or outer diameters are not uniform. For example, one or more portions of the stent may have a tapered outer diameter. The main body may be tapered, the side branch may be tapered or both may be tapered.

The inventive stents may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The inventive stents may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto. Alternatively, the agent may be in at least one indentation on the surface of at least a portion of the stent.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A stent comprising:
a substantially cylindrical tubular body, the tubular body defining a primary lumen, the tubular body comprising a wall and at least one perimeter member defining at least one opening in the wall, the at least one opening having a circumferential length and a longitudinal length, the tubular body having at least one expandable side branch, the at least one side branch comprising a plurality of elongate members, the plurality of elongate members having a first end and a second end, the first end engaged to the at least one perimeter member, the plurality of elongate members having an unexpanded state and an expanded state, in the unexpanded state the plurality of elongate members positioned substantially within the wall of the tubular body, in the expanded state the plurality of elongate members defining a side branch lumen and the second end of the plurality of elongate members extending to a pre-determined distance away from the tubular body, the pre-determined distance the same for all of the plurality of elongate members, the side branch lumen being in fluid communication with the primary lumen.

2. The stent of claim 1, the tubular body having two perimeter members, a first perimeter member defining a first opening, a second perimeter member defining a second opening, the first opening having a center point located at a first longitudinal coordinate and a first circumferential coordinate, and the second opening having a center point located at a second longitudinal coordinate and a second circumferential coordinate.

3. The stent of claim 2, the first longitudinal coordinate different from the second longitudinal coordinate.

4. The stent of claim 1, the circumferential length of the opening greater than the longitudinal length of the opening.

5. The stent of claim 1, wherein the shape of the plurality of elongate members is triangular, columnar or zig-zag.

6. The stent of claim 1, wherein at least one of the plurality of elongate members define at least one hole.

7. The stent of claim 6, having a therapeutic agent within the at least one hole.

8. The stent of claim 7, wherein the therapeutic agent is selected from at least one member of the group consisting of a non-genetic therapeutic agent, a genetic therapeutic agent, cellular material, a polymer agent, and any combination thereof.

9. The stent of claim 1, having four elongate members.

10. The stent of claim 1 wherein the side branch is self-expanding.

11. The stent of claim 1 wherein the side branch is balloon expandable.

12. The stent of claim 1, having twelve elongate members.

13. The stent of claim 1 further comprising at least one radiopaque marker.

14. A bifurcated stent formed by:
providing a stent, the stent comprising an expandable tubular body, the tubular body having a wall, a longitudinal flowpath and at least one perimeter member defining at least one opening in the wall, the tubular body having at least one expandable side branch, the at least one side branch comprising a plurality of elongate members, the plurality of elongate members having a first end and a second end, the first end engaged to the at least one perimeter member, and the second end of the plurality of elongate members extending to a pre-determined distance away from the tubular body, the pre-determined distance the same for all of the plurality of elongate members;
expanding the tubular body of the stent to a first diameter; and
expanding the expandable side branch outward to define a second flowpath which branches off the longitudinal flowpath.

15. The stent of claim 14 wherein at least one of the expanding steps is accomplished with a balloon.

16. The stent of claim 15 wherein at least one of the expanding steps is accomplished by withdrawing a sheath which is disposed over at least a portion of the stent.

17. In combination, a catheter and an expandable stent, the stent having a tubular surface disposed about a longitudinal flowpath, the tubular surface including a plurality of elongate members arranged along the surface about a lateral opening defined by a perimeter member, the plurality of elongate members comprising a one first end and a second end, the first end engaged to the perimeter member, and the second end of the plurality of elongate members extending to a pre-determined distance away from the tubular body, the pre-determined distance the same for all of the plurality of elongate members, the expandable stent disposed about the catheter, the catheter including an elongated catheter member which extends through the opening.

18. An stent comprising an expandable tubular body, the tubular body having a wall and at least one perimeter member defining at least one opening in the wall, the opening having a circumferential length and a longitudinal length, the circumferential length of the opening greater than the longitudinal length of the opening, the stent further comprising an expandable side branch, the expandable side branch comprising a plurality of elongate members, the plurality of elongate members having a first end, a second end and an expanded state, the first end engaged to the at least one perimeter member, the second end extending to a pre-determined distance away from the tubular body in the expanded state, the pre-determined distance the same for all of the plurality of elongate members.
